(19) United States
(12) Patent Application Publication
(10) Pub. No.: US 2006/0234323 A1
(43) Pub. Date: Oct. 19, 2006

(54) Luminescent ATP detection with extended linearity

(76) Inventors: James J. Cali, Verona, WI (US); Dongping Ma, Madison, WI (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/108,621

(22) Filed: Apr. 18, 2005

(51) Int. Cl. C12Q 1/66

(52) U.S. Cl. 435/8

(57) ABSTRACT

A luminescent assay capable of detecting a wider range of ATP concentrations is provided.

### BACKGROUND

Luciferases are found in a wide variety of organisms including fireflies, photobacteria, jellyfish and many others. Luciferases are enzymes which catalyze the production of light by oxidizing a substrate, e.g., luciferin to oxyluciferin, in a process known generally as bioluminescence. The production of photons by luciferase occurs through a two step reaction which consumes luciferin, adenosine triphosphate (ATP) and O₂. In the first step, luciferase catalyzes the formation of luciferyl adenylate from luciferin and ATP. In this first step, pyrophosphate is released and a Mg²⁺ cofactor or another divalent cation is required for proper luciferase function. Upon formation, luciferyl adenylate remains within the active site of luciferase. In the next step, luciferase oxidizes luciferyl adenylate to an electronically excited oxyluciferin with the consumption of oxygen. Light production occurs when the electronically excited oxyluciferin decays to the ground state oxyluciferin. The decay from the excited state to the ground state occurs with the concomitant emission of a photon. The color of the light produced differs with the source of the luciferase and appears to be determined by differences in the structure of the various luciferases. The overall reaction is as follows:

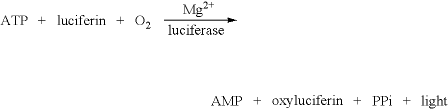

In the presence of non-limiting concentrations of D-luciferin and Mg²⁺ the ATP dependence of the reaction displays Michaelis-Menton saturation kinetics with a characteristic half maximal (Kₘ) and maximal (Vₘₐₓ) reaction rate. In a range of the luciferase-ATP dose curve where ATP is limiting, a change in ATP concentration can be detected as a change in the amount of luminescence generated. It is particularly convenient to measure ATP concentrations in the linear or pseudo-linear portion of the ATP dose curve (at or below Kₘ). Here luminescence changes in direct proportion to ATP concentration (e.g., a doubling in ATP results in a doubling in luminescence).

Thus, one way to detect the presence of ATP in a sample is to employ the ATP-driven luciferase/luciferin reaction. Although luciferase can be used to detect vanishingly small amounts of ATP, a luciferase-based assay for detecting ATP concentrations is limited at the high end of the ATP dose curve (e.g., in the saturated range of the ATP curve) as a change in ATP concentration at the high end of the ATP dose curve results in no change in luminescence.

What is needed is a bioluminescent assay to detect higher concentrations of ATP in a sample, e.g., without diluting the sample.

### SUMMARY OF THE INVENTION

The invention provides luminescent compositions and methods to detect analytes such as adenosine triphosphate (ATP) or ATP consuming enzymes in a sample, such as a biological sample, at ATP concentrations greater than 0.05 mM, e.g., greater than 0.1 mM, by increasing the ATP Kₘ of a beetle luciferase, e.g., a firefly luciferase. The compositions and methods of the invention, by increasing the linear range for ATP detection in a beetle luciferase reaction, provide for measuring moderate ATP concentrations, e.g., from 0.05 mM to 5 mM, in samples such as biological samples, including physiological samples.

As described herein, agents including beetle luciferase inhibitors such as inorganic pyrophosphates (e.g., PPi) and adenosine derivatives can extend the linear range for ATP detection in a luminescent assay. For instance, adenosine monophosphate (AMP), adenosine 5′-O-thiomonophosphate (5′-AMPS), adenosine 5′-[β-thio]diphosphate (ADPβS) and deoxyadenosine triphosphate were competitive inhibitors of a thermostable beetle luciferase (see U.S. Pat. No. 6,602,677, which describes numerous thermostable luciferases including one referred to as Luc146-1H2) mediated luminogenic reaction, as they each increased the luciferase Kₘ for ATP, e.g., by 10 fold. Inhibition by PPi of a thermostable luciferase in a luminogenic reaction was apparently noncompetitive, as PPi decreased the Vₘₐₓ without significantly affecting (2 fold or less) the luciferase Kₘ for ATP, while inhibition by PPi of a native beetle luciferase in a luminogenic reaction was competitive in the presence of certain divalent metals. Surprisingly, a combination of PPi and AMP, 5′-AMPS or ADPβS in a firefly luciferase reaction mixture resulted in a synergistic increase in the luciferase Kₘ for ATP, values being many times higher in the presence of PPi than without PPi. Thus, the luciferase Kₘ for ATP can be increased from a low native level (typically 10 to 20 μM) to a much higher level (up to about 30 mM or 40 mM) to extend the linear range for ATP detection. Accordingly, by varying the concentration of one or more inhibitors of ATP in a beetle luciferase luminogenic reaction, a linear range for ATP detection can be selected. In particular, the Kₘ increase from the combination of PPi and an adenosine derivative is significantly greater than what was achieved with an adenosine derivative alone. The fold increase in the linear range for ATP detection by luciferase is approximately the same as the fold increase in Kₘ that occurs in the presence of PPi and an adenosine derivative. Moreover, for some reactions, the use of a divalent cation other than Mg, e.g., Ca, and one inhibitor of ATP, e.g., AMP, in a beetle luciferase luminogenic reaction can yield a luminogenic reaction mixture capable of extending the linear range for ATP detection.

The invention thus provides a method to directly or indirectly detect the presence or amount of an analyte in a sample. In one embodiment, the invention provides a method to detect the presence or amount of ATP in a sample. The method includes contacting a sample, e.g., one suspected of having ATP, a beetle luciferase reaction mixture, and at least one, e.g., two or more, inhibitor(s) of ATP, to yield a luminogenic reaction mixture. In one embodiment, the inhibitor may be a noncompetitive inhibitor of ATP. In one embodiment, the inhibitor may be an inorganic pyrophosphate, e.g., PPi or a derivative thereof such as μ-monothiopyrophosphate (Halkides et al., Biochemistry, 30:10313 (1991)). In one embodiment, the inhibitor may be a competitive inhibitor of ATP. In one embodiment, the inhibitor may be an adenosine derivative. In one embodiment, a luminogenic reaction mixture contains more than one inhibitor, where one of the inhibitors is a noncompetitive inhibitor of ATP and the other is a competitive inhibitor of ATP. In one embodiment, an adenosine derivative which is a competitive inhibitor of ATP and optionally another inhibitor of ATP is/are contacted with a beetle luciferase reaction mixture before contact with the sample. In another embodiment, the adenosine derivative which is a competitive inhibitor and optionally another inhibitor is/are contacted with the sample before contact with the beetle luciferase reaction mixture. As used herein, a “beetle luciferase reaction mixture” includes reagents for a beetle luciferase luminogenic reaction, for instance, a beetle luciferase such as a Photinus pyralis, Pyrophorus plagiophthalamus or Photuris pennsylvanica luciferase, including recombinant beetle luciferases such as recombinant thermostable and/or mutant beetle luciferases, an appropriate luciferase substrate, e.g., D-luciferin or modified luciferin substrates such as aminoluciferin, and a divalent cation. In one embodiment, a beetle luciferase reaction mixture may substantially lack ATP, e.g., have less than about 0.01 pg (less than 2×10⁻¹⁷ moles) of ATP, so that the ATP for the beetle luciferase luminogenic reaction is generally provided by the sample. However, in another embodiment a beetle luciferase reaction mixture may contain more than 0.01 pg of ATP and in that instance, the amount of luminescence from a control luminogenic reaction, i.e., one without a test sample, may be subtracted from the luminescence in a luminogenic reaction that includes the test sample, for instance, so as to quantitate the ATP in the sample. When present, the amount of the adenosine derivative which is a competitive inhibitor of ATP in the luminogenic reaction mixture is effective to increase the luciferase Kₘ for ATP, for instance, by at least 1.5, 2, 5, 10, 100 or 1000 fold, or greater, relative to the luciferase Kₘ for ATP in the absence of the adenosine derivative. In one embodiment, the presence of two or more inhibitors of ATP, e.g., a noncompetitive and a competitive inhibitor of ATP, in the luminogenic reaction mixture synergistically increases the luciferase Kₘ for ATP. The presence or amount of ATP in the sample is detected or determined, e.g., by detecting or determining luminescence. ATP concentrations of from 0.05 to 5 mM, including from 0.1 mM to 5 mM and 0.25 mM to 5 mM, and up to about 40 mM, in a sample can be measured using the methods and compositions of the invention.

The invention also provides a method to detect the presence or amount of an analyte in an ATP consuming enzymatic reaction. In one embodiment, the method includes detecting the presence, amount or activity of a non-beetle luciferase ATP consuming enzyme in a sample. The method includes contacting a sample having a reaction mixture for a non-beetle luciferase ATP consuming enzyme, which sample may optionally include other components including a cell lysate or subcellular fraction, a beetle luciferase reaction mixture, and one or more inhibitors of ATP, for instance, two inhibitors of ATP, in a beetle luciferase reaction, to yield a luminogenic reaction mixture. In one embodiment, the inhibitor may be a noncompetitive inhibitor of ATP. In one embodiment, the inhibitor may be an inorganic pyrophosphate. In one embodiment, the inhibitor may be a competitive inhibitor of ATP. In one embodiment, the inhibitor may be an adenosine derivative. In another embodiment, where two inhibitors of ATP are employed in a luminogenic reaction, one is a noncompetitive inhibitor of ATP and the other is an adenosine derivative which is a competitive inhibitor of ATP. As used herein, a “reaction mixture for a non-beetle luciferase ATP consuming enzyme” includes the non-beetle luciferase ATP consuming enzyme and optionally includes one or more other reagents for the non-beetle luciferase ATP consuming enzyme reaction, e.g., ATP and/or a substrate for the non-beetle luciferase ATP consuming enzyme may be present in the sample or added to the sample. The ATP in the reaction mixture for the non-beetle luciferase ATP consuming enzyme is present in an amount which, when subsequently combined with a beetle luciferase reaction mixture that does not include one or more inhibitors of ATP, is outside the linear range for a beetle luciferase mediated reaction. When present, the amount of a competitive inhibitor such as an adenosine derivative in the luminogenic reaction mixture is effective to increase the luciferase Kₘ for ATP relative to the luciferase Kₘ for ATP in the absence of the competitive inhibitor. The presence or amount of luminescence is detected or determined. In one embodiment, the presence of two inhibitors, e.g., a noncompetitive and a competitive inhibitor of ATP, in the luminogenic reaction mixture synergistically increases the luciferase Kₘ for ATP. Optionally, the presence or amount of luminescence is compared to the luminescence in a corresponding luminogenic reaction that lacks the non-beetle luciferase ATP consuming enzyme or a corresponding luminogenic reaction that includes the non-beetle luciferase ATP consuming enzyme and an effective amount of an inhibitor of the non-beetle luciferase ATP consuming enzyme.

A non-beetle luciferase ATP consuming enzyme within the scope of the invention is one which has a higher Kₘ for ATP than one or more beetle luciferases. In one embodiment, to detect a non-beetle luciferase ATP consuming enzyme at a non-limiting ATP concentration (Vₘₐₓ), the non-beetle luciferase ATP consuming enzyme has a Kₘ for ATP that is at least 5, e.g., at least 10, fold higher than a beetle luciferase Kₘ for ATP (in the absence of the inhibtor(s)). In another embodiment, to detect the non-beetle luciferase ATP consuming enzyme at its Kₘ ATP concentration, the non-beetle luciferase ATP consuming enzyme has a Kₘ for ATP that is no more than 2 fold higher than a beetle luciferase Kₘ for ATP (in the absence of the inhibtor(s)). In one embodiment, the non-beetle luciferase ATP consuming enzyme has a Kₘ for ATP of about 0.01 mM to about 3 mM. In one embodiment, the non-beetle luciferase ATP consuming enzyme is a membrane bound protein. In another embodiment, the non-beetle luciferase ATP consuming enzyme is an adenylyl cyclase. In yet another embodiment, the non-beetle luciferase ATP consuming enzyme is a kinase, e.g., protein kinase A, protein kinase C, or PI3 kinase, and includes a tyrosine kinase, calcium/calmodulin protein kinase, DNA dependent-protein kinase, or cdc2 kinase. The non-beetle luciferase ATP consuming enzyme in the sample may be isolated or purified non-beetle luciferase ATP consuming enzyme.

In one embodiment, the invention provides a method to detect the activity of a non-beetle luciferase ATP consuming enzyme in a sample. The method includes providing a sample comprising a reaction mixture for a non-beetle luciferase ATP consuming enzyme. That reaction mixture is then contacted with a beetle luciferase reaction mixture, and at least one competitive inhibitor of ATP in a beetle luciferase reaction, so as to yield a luminogenic reaction mixture. The at least one inhibitor is present in an amount that results in the beetle luciferase Kₘ for ATP being the same or greater than the non-beetle luciferase ATP consuming enzyme Kₘ for ATP. Then the presence or amount of luminescence in the luminogenic reaction mixture is detected or determined.

Also provided is a method to detect one or more modulators, e.g., one or more inhibitors or activators or reaction conditions such as pH or ionic strength, that alter the activity of a non-beetle luciferase ATP consuming enzyme. The method includes comparing luminescence from a first luminogenic reaction mixture which includes a reaction mixture for a non-beetle luciferase ATP consuming enzyme that has the enzyme and one or more compounds and/or conditions to be tested, and a beetle luciferase reaction mixture comprising at least one inhibitor of ATP, for example, a noncompetitive inhibitor of ATP and/or a competitive inhibitor of ATP such as an adenosine derivative, with luminescence from a second luminogenic reaction mixture comprising a reaction mixture for a non-beetle luciferase ATP consuming enzyme that includes the enzyme (but not any of the compounds or under test reaction conditions) and a beetle luciferase reaction mixture having the corresponding inhibitor(s) of ATP. When present, the amount of the competitive inhibitor, such as an adenosine derivative, in each luminogenic reaction mixture is effective to increase the luciferase Kₘ for ATP relative to the luciferase Kₘ for ATP in the absence of the adenosine derivative, and effective to increase the luciferase Kₘ for ATP so that it is the same or greater than the non-beetle luciferase ATP consuming enzyme Kₘ for ATP. Then it is determined whether the one or more compounds and/or reaction conditions alter luminescence in the first luminogenic reaction relative to the second luminogenic reaction. In one embodiment, the presence of two inhibitors of ATP, e.g., a noncompetitive and a competitive inhibitor of ATP, in each luminogenic reaction mixture synergistically increases the luciferase Kₘ for ATP. A relative increase in luminescence in the first luminogenic reaction mixture is indicative of an inhibitor of the non-beetle luciferase ATP consuming enzyme, and a relative decrease is indicative of an activator. In one embodiment, the beetle luciferase is a thermostable beetle luciferase. In one embodiment, the beetle luciferase is a native beetle luciferase.

In one embodiment, the invention provides a kit. The kit includes a composition comprising at least two inhibitors of ATP in a beetle luciferase luminogenic reaction, such as an adenosine derivative which is a competitive inhibitor of ATP and a noncompetitive or another competitive inhibitor of ATP. The composition is optionally disposed in a suitable container, and optionally, the composition or kit includes at least one of a beetle luciferase, a beetle luciferase substrate, a non-beetle luciferase ATP consuming enzyme and/or a substrate for the non-beetle luciferase ATP consuming enzyme. The composition may be lyophilized or an aqueous solution.

Also provided is a method to identify a concentration of a divalent cation that alters the Kₘ of a luciferase for ATP. The method includes providing the Kₘ of one or more luciferases for ATP in a luminogenic reaction at one or more concentrations of a plurality divalent metals, optionally in the absence of an inhibitor of ATP. Then a concentration of at least one divalent metal and at least one luciferase is identified having a selected Kₘ for ATP in the luminogenic reaction.

### BRIEF DESCRIPTION OF THE FIGURES

FIGS. 1A-D show relative luminescent units (RLU) for increasing ATP concentrations in the absence (NT; panel A) or presence of luciferase inhibitors (panels B-D).

FIG. 2 illustrates P-glycoprotein (Pgp)-dependent changes in luminescence associated with ATP consumption by the Pgp ATPase. Membrane preparations provided Pgp. The impact of verapamil (a Pgp ATPase activating drug) on Pgp ATPase activity was examined by comparing samples with and without 100 μM Na₃VO₄ and samples plus 300 μM verapamil with and without Na₃VO₄. Na₃VO₄ is a selective inhibitor of Pgp. Thus, the difference in luminescence between samples without verapamil minus and plus Na₃VO₄ (ΔRLU_{basal}) reflects basal Pgp ATP consumption (ATPase activity), and the luminescence between samples with verapamil minus and plus Na₃VO₄ (ΔRLUᵥₑᵣ) reflects verapamil-stimulated Pgp ATP consumption. ATP consumption in the presence of Na₃VO₄ is attributed to minor non-Pgp ATPase activities present in the membrane preparation.

FIG. 3 shows ATP standards (STDS) in extended linearity luminescent reactions. An ATP standard curve was performed so that the RLU of the Pgp reactions could be converted to ATP concentrations by comparison to the curve. Pgp ATPase reaction mixtures without MgATP were incubated at 37° C. in parallel with the Pgp reactions shown in FIGS. 2, 4 and 5. After addition of the thermostable luciferase reaction mixture to these standard samples, MgATP was added to the final concentrations shown on the graph. Samples were then moved to room temperature (about 23° C.) along with the Pgp reactions for 20 minutes before luminescence was read on a plate reading luminometer. Linear regression analysis was performed and the ATP concentrations of the Pgp reactions were interpolated by comparison to the standard curve.

FIG. 4 illustrates basal and verapamil-stimulated Pgp ATPase activity. RLU values from the Pgp ATPase reactions shown in FIG. 2, without verapamil and with 300 μM verapamil, were compared to the standard ATP curve (FIG. 3) to determine their ATP concentrations. From these ATP concentrations, the amounts of ATP consumed by Pgp and specific activities (pmoles ATP consumed/μg Pgp/minute) were calculated.

FIG. 5 shows dose dependence of Pgp ATPase stimulation by verapamil. RLU values from Pgp ATPase reactions without verapamil and with the verapamil concentrations shown on the graph were compared to the standard ATP curve (FIG. 3) to determine their ATP concentrations. From these ATP concentrations, the amounts of ATP consumed by Pgp and specific activities (pmoles ATP consumed/μg Pgp/minute) were calculated.

FIG. 6A shows RLU over time for Pgp ATPase in the presence of verapamil or in the presence of vanadate and verapamil. Recombinant human Pgp expressed in insect cells and prepared as a plasma membrane fraction was exposed to 4 mM ATP at 37° C. for 20 minutes. Pgp ATPase assays were performed as described for FIGS. 2-5. Luminescence was measured immediately after adding the thermostable luciferase to the reaction mixture and thereafter at 5 minute intervals for 65 minutes. Control membranes are a plasma membrane fraction without Pgp from the insect cell expression system used for recombinant human Pgp expression. By comparing samples plus and minus vanadate, ATP consumption plus and minus Pgp ATPase activity can be measured.

FIG. 6B illustrates the use of 10 mM imidodiphosphate (IDP) to stabilize luminescent signals from Pgp ATPase assays.

### DETAILED DESCRIPTION OF THE INVENTION

Definitions

“Analyte” is a substance to be detected in a test sample, but as used herein does not include a beetle luciferase or its corresponding luciferin substrate. Luminescent reactions can be used to detect and quantify analytes such as proteases, lipases, phosphatases, peroxidases, ATPases, glycosidases, and various metabolites such as ATP or NADH.

As used herein, a “luminogenic assay” includes any assay that generates light based on the presence of a particular molecule and which employs a beetle luciferase. A luminogenic assay may directly or indirectly detect, e.g., measure, the amount or presence of another molecule (an analyte). For instance, in one embodiment, a beetle luciferase and an appropriate luciferin substrate may be employed in a luminogenic assay to detect ATP, while in another embodiment, a beetle luciferase and an appropriate luciferin substrate may be employed in a luminogenic assay to detect an analyte which generates or consumes ATP.

The term “quench” as used herein means to inhibit or prevent an enzyme catalyzed reaction, which inhibition or prevention may occur either directly or indirectly. Agents that can be used to quench a reaction are known as “quenching agents.” Agents which selectively quench a reaction are those which, at least one concentration, quench one reaction but not all reactions. For instance, in one embodiment, a selective quench agent is present in an amount which inhibits a non-beetle luciferase catalyzed reaction but has substantially no quenching effect on a beetle luciferase catalyzed reaction.

An “adenosine derivative” includes a mono-, di- or tri-phosphate nucleotide, having adenine or a modified adenine, e.g., azidoadenine, as a base, and a sugar including a ribose, a deoxyribose, or modifications thereof, such as a methoxy group (MeO) at the 2′ position of ribose.

The term “isolated” when used in relation to an enzyme refers to a molecule that is identified and separated from at least one contaminant with which it is ordinarily present. Thus, an isolated enzyme is present in a form or setting that is different from that in which it is found in nature.

As used herein, “substantially pure” or “purified” means an object species is the predominant species present (i.e., on a molar basis it is more abundant than any other individual species in the composition or on the basis of the activity of the object species), and preferably a substantially purified fraction is a composition wherein the object species comprises at least about 50 percent (on a molar basis) of all macromolecular species present. Generally, a substantially pure or purified composition will comprise more than about 80 percent of all macromolecular species present in the composition, more preferably more than about 85%, about 90%, about 95%, and about 99%. Most preferably, the object species is purified to essential homogeneity (contaminant species cannot be detected in the composition by conventional detection methods) wherein the composition consists essentially of a single macromolecular species.

The invention provides compositions and methods which extend the utility of existing luciferase-based luminescent ATP detection technology for use to detect higher levels of ATP or an analyte in an ATP consuming enzymatic reaction. For instance, such compositions and methods are useful for measuring higher ATP levels in a sample without a dilution step to bring ATP levels into the linear range of the detection system. The compositions and methods are also useful to detect or determine the amount or activity of an analyte such as an enzyme that consumes ATP, e.g., an enzyme with a Kₘ for ATP that is similar to or higher than the Kₘ of a beetle luciferase for ATP. In one embodiment, the Kₘ of the non-beetle luciferase for ATP is at least 10 μM, 100 μM, 500 μM, 1 mM, 5 mM, 10 mM or more. Exemplary non-beetle luciferase enzymes include P-glycoprotein, adenylyl cyclase and kinases as well as other ATP-dependent transporters and ion pumps. It is generally desirable to assay non-beetle luciferase ATP consuming enzymes at a non-limiting concentration of ATP, which may be outside the linear range of the luciferase dose response to ATP. By employing one or more beetle luciferase inhibitors which are noncompetitive inhibitors of ATP and/or competitive inhibitors of ATP in an amount effective to increase the luciferase Kₘ for ATP, in the beetle luciferase reaction, the linear range for ATP detection can be adjusted to encompass higher ATP concentrations including ATP saturation concentrations for enzymes of interest. Thus, using a beetle luciferase ATP detection system and one or more inhibitors of ATP, higher concentrations of ATP and/or the activity of enzymes of interest may be detected. For example, using a beetle luciferase ATP detection system and one or more inhibitors of ATP, the activity of enzymes that consume ATP may be observed as a decrease in the luminescent signal that parallels their consumption of ATP at ATP concentrations that are higher than the linear range for luciferase.

In one embodiment, to detect the presence or amount of ATP in a sample, a luminogenic reaction mixture is prepared. The luminogenic reaction mixture contains a sample, a beetle luciferase reaction mixture, and at least one, e.g., at least two, inhibitors of ATP. In one embodiment, the luminogenic reaction mixture contains a noncompetitive inhibitor of ATP and/or a competitive inhibitor of ATP. In another embodiment, the luminogenic reaction mixture contains at least two competitive inhibitors of ATP. The sample may be a cellular sample, a cell lysate, a subcelluar fraction such as a membrane fraction, S10, S100 or S150 fraction, isolated or purified ATP, a physiological fluid sample, or an in vitro reaction, for instance, an in vitro transcription or in vitro transcription/translation mixture, or purified enzyme, such as a purified ATPase or kinase. The sample may be mixed with one or more of the inhibitor(s), including noncompetitive and/or competitive inhibitors, prior to mixing with the beetle luciferase reaction mixture. Alternatively, one or more of the inhibitors, e.g., noncompetitive and/or competitive inhibitor(s), may be mixed with the beetle luciferase reaction prior to mixing with the sample. In yet another embodiment, the sample, beetle luciferase reaction mixture and one or more of the inhibitors, for instance, a noncompetitive and/or competitive inhibitor(s), is/are combined at the same time. Luminescence is then detected or determined. Luminescent measurements may be made at one or more time points optionally beginning prior to contact of any of the sample, beetle luciferase reaction mixture and one or more of the inhibitors, or with two or fewer of the sample, beetle luciferase reaction mixture and one or more of the inhibitors, including noncompetitive and/or competitive inhibitors (as a control), and at any time (one or more time points) after the sample, beetle luciferase reaction mixture and/or one or more of the inhibitors are combined. In one embodiment, various concentrations of ATP are each combined with a beetle luciferase reaction mixture and one or more of the inhibitors to prepare a standard curve. Such a curve may be used to determine the ATP concentration in a sample, e.g., a cell lysate. As luminescence in the extended linearity ATP assay described herein is proportional to ATP concentration, the ATP concentration in the sample, which may include moderate ATP concentrations even after dilution with the beetle luciferase reaction mixture and the inhibitor(s), can be determined. In one embodiment, at least two inhibitors are employed in the assay, where one inhibitor is a competitive inhibitor of ATP in the beetle luciferase reaction, e.g., an adenosine derivative and the other is an inorganic pyrophosphate such as PPᵢ. The amount of the inhibitor(s) is effective to increase the beetle luciferase Kₘ for ATP relative to the luciferase Kₘ for ATP, for instance, by at least 1.5 fold relative to the luciferase Kₘ for ATP, in the absence of the inhibitor(s).

A luminescent reaction mixture suitable for an increased linear range for ATP detection can be used to measure ATP consumption by an ATPase in which a moderate or high ATP concentration leads to maximal activity (i.e., an enzyme with a moderate or high Kₘ for ATP). One example of such an enzyme is the multi-drug transporter P-glycoprotein (Pgp or MDR1). Pgp is an ATP-dependent efflux pump for a wide range of drugs and plays an important role in multi-drug resistance and certain adverse drug-drug interactions. Drugs that are transported by Pgp can be identified as stimulators of its ATPase activity. Four to 5 mM ATP results in maximal Pgp ATPase activity and that concentration of ATP is higher than the linear range of a beetle luciferase, for instance, a thermostable luciferase, for ATP detection. By adding an adenosine derivative, such as AMP, and PPi to the beetle luciferase reaction mixture, the linear range may be extended to include higher ATP concentrations for maximal activity of the non-beetle luciferase ATPase. A Pgp ATPase assay may be conducted by adding first ATP to a sample having, or suspected of having, the Pgp ATPase. Generally, a positive control sample includes isolated Pgp, which is typically provided as a membrane fraction from cells that express Pgp. The non-beetle luciferase ATP consuming assay may be incubated for any period of time, e.g., from 0 minutes to one or more hours. This first reaction is then combined with a beetle luciferase reaction mixture containing one or more inhibitors of ATP, e.g., noncompetitive and/or competitive inhibitors of ATP, and optionally one or more quenchers of the non-beetle luciferase ATP consuming reaction, to provide for an extended linear range. Optionally, the non-beetle luciferase ATP consuming enzyme reaction is quenched prior to the addition of the inhibitor(s), including noncompetitive and/or competitive inhibitors, and/or the beetle luciferase reaction mixture. The beetle luciferase reaction mixture may also optionally contain one or more pyrophosphatase inhibitors. ATP consumption by Pgp is detected as a decrease in luminescence from the beetle luciferase reaction, relative to samples that lack Pgp ATPase activity. Drugs that stimulate Pgp ATPase may yield a larger decrease in luminescence relative to samples without drug. Since P-glycoprotein is an ATP-dependent drug efflux pump that influences the disposition of numerous drugs, substrates and inhibitors of that glycoprotein may also be identified by their effects on its ATPase activity.

A similar assay may be used for other ATPases. In one embodiment, an increased linear range for ATP detection can be used to measure ATP consumption by a kinase with a relatively high ATP concentration for maximal activity (i.e., an enzyme with a high Kₘ for ATP). In yet another embodiment, an increased linear range for ATP detection can be used to measure ATP consumption by an adenylyl cyclase with a relatively high ATP concentration for maximal activity. The assay may also be used to identify or detect substrates, activators and/or inhibitors of those ATPases.

The ATP or non-beetle luciferase ATP consuming enzyme may be present in a sample, for instance, a sample that comprises cells or is a cell lysate. Cells within the scope of the invention include prokaryotic and eukaryotic cells, including plant cells and vertebrate cells, for instance, mammalian cells including, but not limited to, human, non-primate human, bovine, equine, ovine, swine, caprine, feline, canine, mink, rodent or avian cells. Generally, a sample comprising cells is treated so as to permeabilize or lyse the cells in the sample. Thus, for detection of ATP, or a cytosolic or other enzyme not present on the surface of the cell or otherwise not accessible to assays reagents, it is preferred to disrupt the cells or subcellular fractions thereof. Methods for permeabilization, lysis or disruption of cells or subcellular fractions thereof are well known in the art. A wide variety of equipment is available for mechanical disruption, including sonicators (ultrasonic generators) and French presses. Cells can be disrupted by osmotic shock, by treatments such as a series of freeze-thaw cycles, or a rapid alteration of the ionic strength of the environment, or by the use of agents that directly disrupt cell membranes such as enzymes like lysozyme or chemical agents such as detergent or surfactants and antibacterials such as polymixin B and chlorhexidine. Such chemical reagents are commercially available and commonly referred to as “extractants”. Typical extractants include general cationic detergents such as CTAB (cetyl trimethyl ammonium bromide), anionic detergents such as sodium dodecyl sulfate and nonionic surfactants such as poloxyethylene alkyl phenyl ethers (e.g., Tritons™, from Sigma, St. Louis, Mo.), Tergitol®, e.g., Tergitol®NP-9, nonoxynols, or other materials such as polymixin B sulfate or chlorhexidine, and proprietary formulae such as Extractant™ (F352A, Promega Corp., Madison, Wis.) and Celsis-Lumac (1290142, Celsis, Evanston, Ill.). A typical concentration for such detergents for use in disrupting cells ranges from about 0.01% to 10.0% in an aqueous solution. Cationic detergents are known to release the contents of eukaryotic cells as well as all other kinds of cells. In contrast, the use of a non-ionic detergent is effective for releasing materials from eukaryotic cells without disturbing other kinds of cells. Thus, one can distinguish between bacterial cells versus eukaryotic cells.

In one embodiment, the sample contains isolated or purified ATP or isolated or purified non-beetle luciferase ATP consuming enzyme, e.g., the sample is a subcellular fraction such as a membrane fraction.

The luciferase/luciferin reaction is well known in the art, and there are commercial sources for the necessary reagents as well as protocols for their use. For example, several luciferase/luciferin reagents along with luciferase are available from Promega Corp., Madison, Wis. Commercially available luciferases include wild-type and recombinant luciferases, including luciferases modified in vitro, e.g., by mutagenesis or recombination. In one embodiment, thermostable luciferases may be employed. Thermostable luciferases are generally resistant to the destabilizing effect of materials used to permeabilize the cells, and some contaminating activity may be reduced by heat denaturation.

The invention will be described by the following non-limiting examples.

### EXAMPLE 1

### Use of Two Different Luciferases in a Luminescent Extended Linearity ATP Assay

To formulate a beetle luciferase luminescent reaction mixture that results in an increase in the Kₘ and saturation concentration for ATP, thereby increasing the linear ATP detection range, potential ATP competitive luciferase inhibitors were tested. A competitive enzyme inhibitor is likely to increase the Kₘ for the substrate with which it competes without affecting Vₘₐₓ. Adenosine derivatives and PPi were tested as luciferase inhibitors and for their effects on a beetle luciferase Kₘ for ATP.

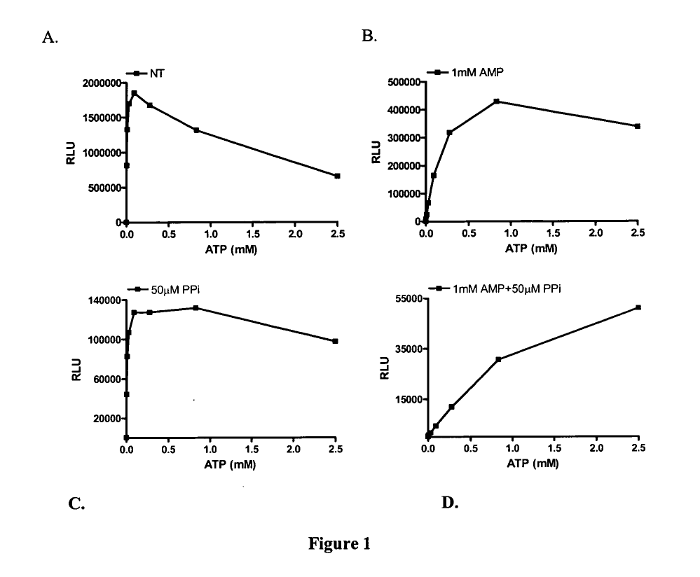

Materials and Methods The reaction mixture for data shown in FIG. 1 and Table 1 contained 0.2 mg/ml of a thermostable luciferase (see U.S. Pat. No. 6,602,677; Luc146-1H2), 20 mM Na₃Citrate (pH 6.0), 55 mM morpholinoethanesulfonic acid (pH 6.0), 11 mM MgSO₄, 2.5 mM (4S)-4,5-dihydro-2-(6-hydroxybenzothiazolyl)-4-thiazolecarboxylic acid (D-luciferin), 0.6 mM 1,2-cyclohexanediaminetetraacetic acid (CDTA), 1% porcine collagen (Prionex®) and from 0 to 2.5 mM adenosine triphosphate (ATP). When present, AMP and PPi were at 1 mM and 50 μM, respectively. The reactions were performed at room temperature (23° C.).

Results

The presence of AMP and PPi in a firefly luciferase reaction mixture resulted in relative luminescent units (RLU) which increased in a linear fashion with increases in ATP concentration. For FIG. 1 and Table 1, thermostable luciferase (a recombinant mutant form of luciferase from Photuris pennsylvanica) reactions were performed at ATP concentrations ranging from 0 to 2.5 mM with the indicated additions of 1 mM AMP and 50 μM PPi, or in the absence of AMP and PPi (NT). Linear regression was performed for the full range of ATP concentrations and various sub-ranges of ATP concentrations. R² values are given in Table 1 as a quantitative measure of linearity. The data was also fit to a hyperbolic function (Y=Vₘₐₓ·X/(Kₘ+X), where X=[ATP], Y=RLU) with calculations of Kₘ for ATP. Kₘ(μM) values from the hyperbolic curve fits are shown in parenthesis. This analysis revealed that improvements to linearity correlated positively with increased Kₘ and suggested that AMP and PPi act synergistically to improve linearity by synergistically increasing the luciferase Kₘ for ATP.

TABLE 1
Calculated R² and Kₘ* values

| ATP range | NT^{a} | AMP^{b} | PPi^{c} | AMP^{b}/PPi^{c} |
|---|---|---|---|---|
| 0 to 2.5 mM | 0.059 (1) | 0.425 (115) | 0.059 (5) | 0.935 (1449) |
| 0 to 0.8 mM | 0.046 (3) | 0.838 (203) | 0.304 (6) | 0.997 (2926) |
| 0 to 0.3 mM | 0.265 (4) | 0.966 (243) | 0.418 (6) | 0.999 (1606) |
| 0 to 0.09 mM | 0.531 (4) | 0.994 (232) | 0.625 (6) | 0.999 (330) |

^{a}NT = no AMP, no PPi
^{b}AMP = 1 mM
^{c}PPi = 50 μM
*Kₘ values are in parentheses (μM)

In addition to the AMP and PPi combination, combinations of PPi with adenosine 5′-[β-thio] diphosphate (ADPβS), adenosine 5′-O-thiomonophosphate (5′AMPS) and deoxyadenosine triphosphate (dATP) also acted synergistically to improve linearity and increase the thermostable luciferase Kₘ for ATP (Table 2). This demonstrates that, in addition to AMP, other adenosine derivatives, including di- and tri-phosphates, can synergize with PPi in a luciferase assay to improve linearity for ATP detection. Data for Table 2 was derived as described above except the range of ATP concentrations was from 0 to 1.67 mM. Data for Table 3 includes a range of ATP concentrations from 0 to 1.67 mM and 0 to 2.5 mM.

TABLE 2

| | |
|---|---|
| No PPi | 50 μM PPi |

| | R² | Kₘ(μM) | R² | Kₘ(μM) |
|---|---|---|---|---|
| NT | 0.104 | 2 | 0.038 | 4 |
| 1 mM AMP | 0.534 | 109 | 0.974 | 1704 |
| 2 mM 5′AMPS | 0.456 | 91 | 0.947 | 1074 |
| 2 mM ADP S | 0.437 | 93 | 0.887 | 581 |
| 5 mM dATP | 0.538 | 127 | 0.73 | 232 |

TABLE 3

| | ATP Kₘ(mM) | R² | ATP range |
|---|---|---|---|
| NT | 0.002 | 0.104 | 0-1.67 mM |
| 1 mM AMP | 0.109 | 0.543 | 0-1.67 mM |
| 1 mM AMP + 50 μM PPi | 1.704 | 0.974 | 0-1.67 mM |
| 2 mM AMP + 50 μM PPi | 10.86 | 0.996 | 0-2.5 mM |
| 2 mM AMP + 100 μM PPi | 40.21 | 0.999 | 0-2.5 mM |
| 3 mM AMP + 50 μM PPi | 11.47 | 0.997 | 0-2.5 mM |

In further experiments with the thermostable luciferase, MgSO₄ was replaced with 20 mM MgCl₂, MnCl₂, CaCl₂, CoCl₂, ZnCl₂, SrCl₂ or NiCl₂ (Table 4). There was little difference in basal Kₘ with the different metals. The data in Table 4 show that when non-Mg metals were employed with AMP and PPi, Co and Ni, and to some extent Ca and Sr, resulted in a synergistic increase in the Kₘ. When the non-Mg metals were employed with AMP, some metals increased the Kₘ to a greater extent than Mg, i.e., Mn, Sr, Ca and Ni. When the non-Mg metals were employed with PPi, Ni increased the Kₘ for ATP by about 10 fold. Other combinations of metals and inhibitor(s) had less of an effect.

TABLE 4

| | | | |
|---|---|---|---|
| NT | AMP | PPi | AMP/PPi |

ATP Kₘ (mM)

| | | | | |
|---|---|---|---|---|
| MgSO₄ | 5 | 218 | 8 | 1914 |
| MgCl₂ | 4 | 137 | 4.5 | 344 |
| CoCl₂ | 1.4 | 62 | 4.7 | 1803 |
| NiCl₂ | 2 | 238 | 23 | 6390 |
| CaCl₂ | 1.4 | 1731 | 2 | 1955 |
| SrCl₂ | 7 | 1095 | 6 | 1359 |
| ZnCl₂ | 3 | 45 | 2 | 47 |
| MnCl₂ | 6.5 | 394 | 0.8 | 41 |

R²

| | | | | |
|---|---|---|---|---|
| MgSO₄ | 0.08687 | 0.793 | 0.3222 | 0.9197 |
| MgCl₂ | 0.04314 | 0.7256 | 0.1654 | 0.9183 |
| CoCl₂ | 0.02298 | 0.6226 | 0.309 | 0.9428 |
| NiCl₂ | 0.05097 | 0.8315 | 0.4035 | 0.986 |
| CaCl₂ | 0.06516 | 0.9721 | 0.1604 | 0.9448 |
| SrCl₂ | 0.2236 | 0.9603 | 0.1527 | 0.9698 |
| ZnCl₂ | 0.1376 | 0.3417 | 0.1614 | 0.9076 |
| MnCl₂ | 0.08143 | 0.8594 | 0.1258 | 0.4427 |

The effect of AMP and PPi to synergistically increase Kₘ for ATP and improve linearity for luminescent detection of ATP was not limited to a 10 thermostable luciferase. The effect was also observed with a wild type luciferase from Photinus pyralis (QuantiLum® recombinant luciferase) over a range of ATP concentrations from 0 to 2.5 mM (Table 5). With this enzyme, compared to reactions with no PPi or AMP, PPi alone caused small increases in R² and Kₘ (200 μM PPi caused a 9% increase in R² and 28% increase in Kₘ ) and AMP alone showed a general trend to decrease R² and Kₘ (3 mM AMP caused a 2.5% decrease in R² and 65% decrease in Kₘ). However, the combination of PPi and AMP caused a substantial increase in both R² and Kₘ (200 μM PPi and 3 mM AMP caused 1.64 fold increase in R² and 3.9 fold increase in Kₘ). When MgCl₂, MnCl₂, CaCl₂, CoCl₂, ZnCl₂, SrCl₂ or NiCl₂ replaced MgSO₄ in reactions with QuantiLum® and one or more ATP inhibitors, reactions with Zn and AMP, or Zn, AMP and PPi, as well as reactions with PPi and Co or Ni, increased the Kₘ (Table 6).

TABLE 5*

| | | 0 μM PPi | 50 μM PPi | 100 μM PPi | 200 μM PPi |
|---|---|---|---|---|---|
| 0 mM | R²: | 0.523 | 0.561 | 0.568 | 0.574 |
| AMP | Kₘ(μM): | 105 | 121 | 130 | 146 |
| 1 mM | R²: | 0.599 | 0.779 | 0.823 | 0.854 |
| AMP | Kₘ(μM): | 58 | 215 | 312 | 435 |
| 2 mM | R²: | 0.536 | 0.769 | 0.814 | 0.863 |
| AMP | Kₘ(μM): | 40 | 181 | 262 | 437 |
| 3 mM | R²: | 0.510 | 0.760 | 0.809 | 0.857 |
| AMP | Kₘ(μM): | 37 | 167 | 248 | 413 |

*reactions employed MgATP

TABLE 6

| | | | |
|---|---|---|---|
| NT | AMP | PPi | AMP + PPi |

ATP Kₘ (μM)

| | | | | |
|---|---|---|---|---|
| MgSO₄** | 39 | 15 | 128 | 93 |
| MgCl₂ | 115 | 56 | 276 | 213 |
| CoCl₂ | 314 | 139 | 751 | 506 |
| NiCl₂ | 906 | 1105 | 2417 | 1540 |
| CaCl₂ | 1906 | 980 | 1451 | 1038 |
| MnCl₂ | 120 | 44 | 170 | 110.6 |
| ZnCl₂ | 63 | 585 | 109 | 2393 |
| SrCl₂ | 373 | 110 | 3293 | 2654 |

R²

| | | | | |
|---|---|---|---|---|
| MgSO₄ | 0.2971 | 0.2064 | 0.4898 | 0.5345 |
| MgCl₂ | 0.5554 | 0.4645 | 0.6773 | 0.7009 |
| CoCl₂ | 0.7775 | 0.7008 | 0.8543 | 0.8218 |
| NiCl₂ | 0.8814 | 0.9246 | 0.9679 | 0.9486 |
| CaCl₂ | 0.9667 | 0.9377 | 0.9364 | 0.9285 |
| SrCl₂ | 0.781 | 0.6097 | 0.969 | 0.9745 |
| ZnCl₂ | 0.4249 | 0.9057 | 0.5708 | 0.9763 |
| MnCl₂ | 0.6245 | 0.3881 | 0.6315 | 0.5799 |

**reactions employed NaATP; thus, as ATP increases for measuring Km, the total free Mg decreases as more is bound to ATP

Based on the results in Tables 1-6, by selecting a particular divalent metal and beetle luciferase, the Kₘ of that luciferase for ATP can be selected.

### EXAMPLE II

### Use of a Luminescent Extended Linearity ATP Assay to Detect a Non-Luciferase ATPase

Pgp, also known as MDR1 and ABCB1, is a 170 kDa integral plasma membrane protein that functions as an ATP dependent drug efflux pump. Drugs and other chemicals that are transported by Pgp typically stimulate its ATPase activity. Pgp has a high Kₘ for ATP (about 0.5 to 1.0 mM). This means that for Vₘₐₓ activity, an assay reaction mixture includes about ≧4 mM ATP. The linear range for ATP detection by a beetle luciferase, e.g., a thermostable beetle luciferase, without an extended linearity formulation is approximately ≦50 μM ATP. At ≧4 mM ATP, Pgp would consume ATP outside of the linear range for a beetle luciferase. As described below, adding PPi and AMP to the luciferase reaction mixture, the linearity for ATP detection can be extended for optimal detection of ATP consumption by Pgp.

Materials and Methods

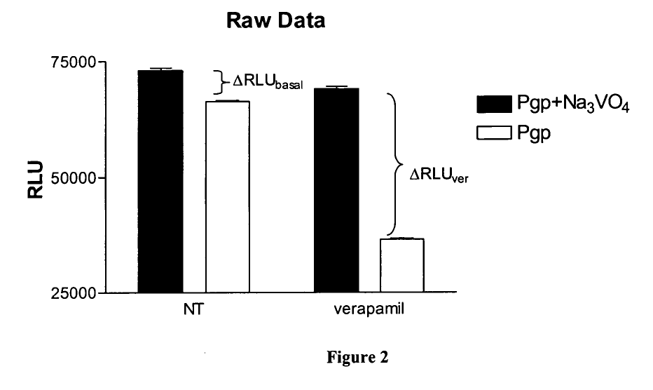

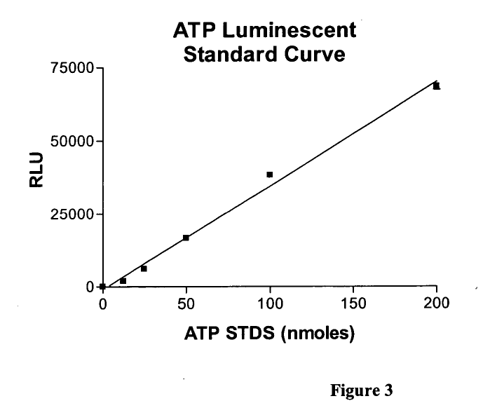

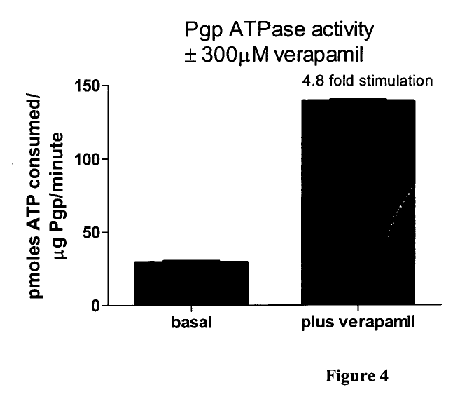

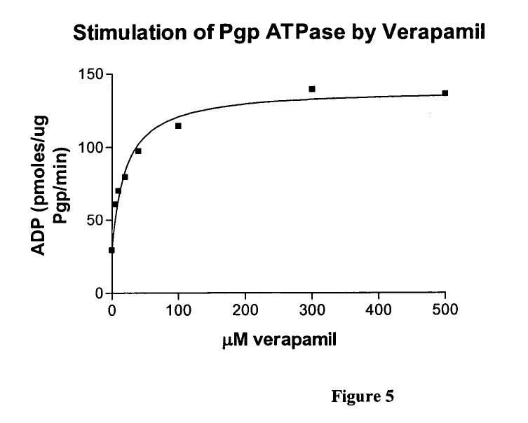

The Pgp ATPase reaction mixture used to obtain the data shown in FIGS. 2-5 contained the following: 660 μg/ml recombinant human Pgp (membrane fraction from insect cell expression system), 50 mM morpholinoethanesulfonic acid (MES), 2 mM ethyleneglycol-bis(2-aminoethylether)-N,N,N′,N′-tetraacetic acid (EGTA), 2 mM dithiothreitol (DTT), 50 mM KCl, 5 mM sodium azide, and Tris-base added to bring the pH to pH 6.8. A mixture of the + and − isomers of verapamil was added at the concentrations indicated. Reaction volumes were 50 μl with 4 mM MgATP added in white, opaque 96 well plates.

Reactions were incubated at 37° C. for 20 minutes before addition of an equal volume (50 μl) of a beetle luciferase reaction mixture. The beetle luciferase reaction mixture used to obtain the data shown in FIGS. 2-5 contained 0.4 mg/ml of a thermostable luciferase, 40 mM Na₃Citrate (pH 6.0), 110 mM morpholinoethanesulfonic acid (MES) (pH 6.0), 22 mM MgSO₄, 4 mM NaF, 20 mM IDP, 5.0 mM (4S)-4,5-dihydro-2-(6-hydroxybenzothiazolyl)-4-thiazolecarboxylic acid (D-luciferin), 1.2 mM 1,2-cyclohexanediaminetetraacetic acid (CDTA), 2% porcine collagen (Prionex®), 2% Tergitol, 2 mM AMP and 100 μM PPi. In the assay scheme for Pgp ATPase activity, the concentrations of all these components are diluted in half with the addition of an equal volume of the thermostable luciferase reaction mixture. Note that both mixtures contained MES, so the final concentration of MES was 80 mM.

Results

A beetle luciferase based ATP detection assay was used to detect Pgp ATPase activity in a membrane fraction that contained Pgp. In the examples shown in FIGS. 2-5, a Pgp ATPase membrane fraction was first exposed to 4 mM ATP in an ATPase reaction mixture. After 20 minutes of ATP consumption by Pgp at 37° C., a luciferase reaction mixture of equal volume was added to the Pgp reaction, bringing the maximal ATP concentration down to 2 mM. The luciferase reaction mixture served to initiate a luminescent reaction and also stop the Pgp reaction by introducing a non-ionic detergent (Tergitol). Because the brightness of luminescence is dependent on ATP concentration, the brightest reaction occurs when no ATP is consumed (control reaction). Luminescence in reactions where ATP has been consumed is decreased relative to the control, and that decrease is in proportion to the amount of ATP consumed. That is, Pgp dependent decreases in luminescence reflect ATP consumption by Pgp. Basal Pgp ATPase activity was observed as a decrease in luminescence and relatively larger decreases were seen with verapamil, a known substrate for transport by Pgp.

### EXAMPLE III

### Luminescent Signal Stability in Extended ATP Assays

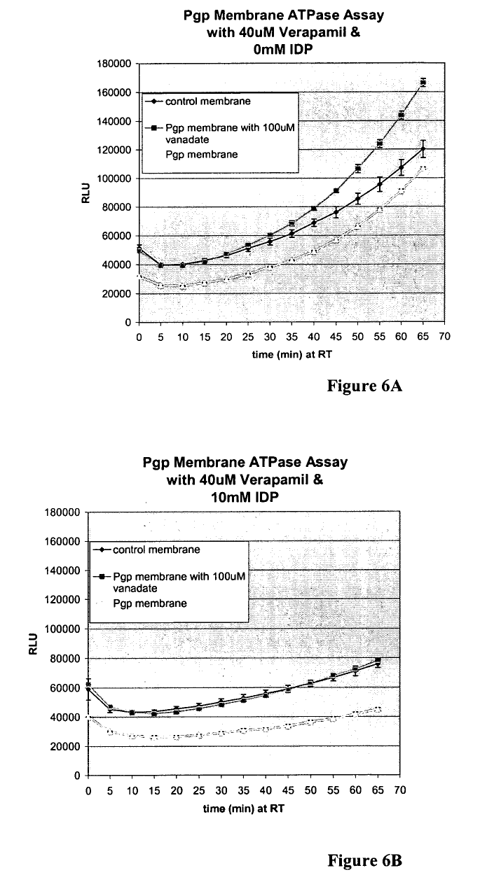

For the Pgp ATPase assays described in the Examples above (FIGS. 2-5), the beetle luciferase reaction mixture was added to samples that were incubating at 37° C. and then the temperature was allowed to equilibrate to room temperature (about 23° C.) for 20 minutes before a luminescent reading was taken. However, when luminescence was read immediately after addition of the beetle luciferase reaction mixture and then repeatedly thereafter at regular time intervals, it was observed that luminescent signals did not remain constant, rather they increased over time (FIG. 6A). This might be due to degradation of PPi by a pyrophosphatase activity(s) present in the Pgp plasma membrane preparation. If PPi were being degraded, this would relieve inhibition of luciferase resulting an in increase in the luminescent signal. To minimize this effect, the pyrophosphatase inhibitor imidodiphosphate (IDP) was added to the luciferase reaction mixture. The IDP substantially improved signal stability by significantly reducing the rate of signal increase (FIG. 6B). Thus, a pyrophosphatase inhibitor such as IDP can be used to stabilize luminescent signals when samples that contain pyrophosphatase are used. The effect of IDP to stabilize luminescent signals over time can be appreciated by comparing FIG. 6A with no IDP to FIG. 6B with 10 mM IDP.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method to detect the presence or amount of ATP in a sample, comprising:
a) contacting a sample, a beetle luciferase reaction mixture, and at least two inhibitors of ATP so as to yield a luminogenic reaction mixture, wherein one of the inhibitors is an adenosine derivative which is present in an amount effective to increase the beetle luciferase Kₘ for ATP relative to the luciferase Kₘ for ATP in the absence of the adenosine derivative, and wherein the amount of the inhibitors in the luminogenic reaction mixture provides for a luminogenic reaction that is capable of detecting an ATP concentration beyond the linear range for a corresponding luminogenic reaction that lacks the at least two inhibitors; and
b) detecting the presence or amount of luminescence in the luminogenic reaction mixture.

2. The method of claim 1 wherein a second inhibitor is a noncompetitive inhibitor of ATP.

3. The method of claim 1 wherein the at least two inhibitors and the sample are contacted with each other before contact with the beetle luciferase reaction mixture.

4. The method of claim 1 wherein the at least two inhibitors and the beetle luciferase reaction mixture are contacted with each other before contact with the sample.

5. The method of claim 1 wherein the Kₘ for ATP is synergistically increased.

6. The method of claim 1 wherein the beetle luciferase Kₘ for ATP is increased by greater than about 1.5 fold relative to the beetle luciferase Kₘ for ATP in the absence of the adenosine derivative.

7. The method of claim 1 wherein luminescence is correlated to the presence or amount of ATP in the sample.

8. The method of claim 1 wherein the sample includes a reaction mixture for a non-beetle luciferase ATP consuming enzyme, and wherein the non-beetle luciferase ATP consuming enzyme Kₘ for ATP is the same or greater than the beetle luciferase Kₘ for ATP in the absence of the inhibitors.

9. A method to detect the activity of a non-beetle luciferase ATP consuming enzyme in a sample, comprising:
a) contacting a sample comprising a reaction mixture for a non-beetle luciferase ATP consuming enzyme, a beetle luciferase reaction mixture, and at least one competitive inhibitor of ATP in a beetle luciferase reaction, so as to yield a luminogenic reaction mixture, wherein the at least one inhibitor is present in an amount that results in the beetle luciferase Kₘ for ATP being the same or greater than the non-beetle luciferase ATP consuming enzyme Kₘ for ATP; and
b) detecting the presence or amount of luminescence in the luminogenic reaction mixture.

10. The method of claim 9 wherein the luminogenic reaction mixture further comprises a second inhibitor of ATP.

11. The method of claim 8 or 10 wherein the inhibitors synergistically increase the beetle luciferase Kₘ for ATP.

12. The method of claim 9 wherein the at least one inhibitor is an adenosine derivative.

13. The method of claim 12 wherein the luminogenic reaction mixture comprises the adenosine derivative and a second inhibitor of ATP.

14. The method of claim 8 or 9 wherein the presence or amount of luminescence in the luminogenic reaction mixture is compared to a corresponding luminogenic reaction mixture which does not include the non-beetle luciferase ATP consuming enzyme or a corresponding luminogenic reaction mixture which includes an effective amount of an inhibitor of the non-beetle luciferase ATP consuming enzyme.

15. The method of claim 8 or 9 wherein the presence or amount of luminescence is correlated to the presence or activity of the non-beetle luciferase ATP consuming enzyme.

16. The method of claim 8 or 9 wherein the non-beetle luciferase ATP consuming enzyme is P-glycoprotein (Pgp), an adenylyl cyclase or a kinase.

17. The method of claim 1 or 9 wherein the beetle luciferase is a thermostable beetle luciferase.

18. The method of claim 1 or 9 wherein the beetle luciferase is a firefly luciferase.

19. The method of claim 1 or 12 wherein the adenosine derivative is AMP, 5′ AMPS, ADPβS or deoxyadenosine triphosphate.

20. The method of claim 1 or 12 wherein the concentration of the adenosine derivative is about 0.1 mM to about 10 mM.

21. The method of claim 1 or 10 wherein one of the inhibitors is PPᵢ.

22. The method of claim 21 wherein the luminogenic reaction mixture further comprises an inhibitor of inorganic pyrophosphatase.

23. The method of claim 21 wherein the concentration of PPᵢ is about 10 μM to 500 μM.

24. The method of claim 8 or 9 wherein the sample comprises isolated non-beetle luciferase ATP consuming enzyme.

25. The method of claim 24 wherein the isolated non-beetle luciferase ATP consuming enzyme is isolated Pgp, isolated adenylyl cyclase or isolated kinase.

26. The method of claim 1 or 9 wherein the sample is a cell lysate.

27. The method of claim 26 wherein the lysed cells are cultured cells.

28. The method of claim 26 wherein the lysed cells are mammalian cells.

29. The method of claim 1 or 9 wherein the sample is a subcellular fraction.

30. A method to detect one or more modulators of a non-beetle luciferase ATP consuming enzyme, comprising:
a) comparing luminescence from a first luminogenic reaction mixture comprising a reaction mixture for a non-beetle luciferase ATP consuming enzyme which comprises the non-beetle luciferase ATP consuming enzyme and one or more compounds and/or reaction conditions to be tested, and a beetle luciferase reaction mixture comprising at least one inhibitor of ATP, to luminescence from a second luminogenic reaction mixture comprising a reaction mixture for the non-beetle luciferase ATP consuming enzyme which comprises the non-beetle luciferase ATP consuming enzyme but lacks the one or more compounds and/or is subjected to different reaction conditions, and a beetle luciferase reaction mixture comprising the at least one inhibitor, wherein the non-beetle luciferase ATP consuming enzyme Kₘ for ATP is greater than the beetle luciferase Kₘ for ATP in the absence of the at least one inhibitor, wherein the at least one inhibitor of ATP in each luminogenic reaction is present in an amount that results in the luciferase Kₘ for ATP being the same or greater than the non-beetle luciferase ATP consuming enzyme Kₘ for ATP; and
b) detecting or determining whether the one or more compounds and/or reaction conditions in the first luminogenic reaction mixture alter the luminescence in the first luminogenic reaction mixture relative to the second luminogenic reaction mixture.

31. The method of claim 30 wherein the at least one inhibitor is an adenosine derivative.

32. The method of claim 30 wherein two inhibitors of ATP are employed.

33. The method of claim 32 wherein the inhibitors synergistically increase the luciferase Kₘ for ATP.

34. The method of claim 30 wherein the non-beetle luciferase ATP consuming enzyme is Pgp, adenylyl cyclase or a kinase.

35. The method of claim 30 wherein the reaction mixture for the non-beetle luciferase ATP consuming enzyme comprises isolated non-beetle luciferase ATP consuming enzyme.

36. The method of claim 30 wherein the reaction mixture for the non-beetle luciferase ATP consuming enzyme comprises a cell lysate.

37. The method of claim 36 wherein the lysed cells are cultured cells.

38. The method of claim 36 wherein the lysed cells are mammalian cells.

39. The method of claim 30 wherein the beetle luciferase reaction mixture further comprises at least one agent in an amount which selectively quenchs the non-beetle luciferase ATP consuming enzyme reaction.

40. The method of claim 39 wherein the one agent is a non-ionic detergent.

41. The method of claim 39 wherein the one agent is an inhibitor of the non-beetle luciferase ATP consuming enzyme reaction.

42. The method of claim 1 or 30 further comprising detecting or determining the presence or amount of another molecule of interest.

43. The method of claim 30 wherein the luminogenic reaction mixture further comprises an inhibitor of inorganic pyrophosphatase.

44. A kit comprising:
a composition comprising at least two inhibitors of ATP in a beetle luciferase reaction, wherein one inhibitor is an adenosine derivative, and
optionally, one or more of the following reagents: isolated beetle luciferase, isolated non-beetle luciferase ATP consuming enzyme, a beetle luciferase substrate and/or a substrate for the non-beetle luciferase ATP consuming enzyme, wherein if present the one or more reagents are optionally separately packaged.

45. The kit of claim 44 wherein the second inhibitor is a noncompetitive inhibitor of ATP.

46. The kit of claim 44 further comprising instructions for conducting a luminogenic reaction in which the ATP inhibitors increase the beetle luciferase Kₘ for ATP.

47. The kit of claim 44 further comprising a suitable container, the composition disposed therein.

48. The kit of claim 44 further comprising a suitable container, the isolated beetle luciferase, the isolated non-beetle luciferase ATP consuming enzyme, the beetle luciferase substrate or the non-beetle luciferase ATP consuming enzyme substrate thereof, disposed therein.

49. The kit of claim 44 further comprising a selective quench reagent for a non-beetle luciferase ATP consuming enzyme reaction.

50. The kit of claim 44 wherein at least one of the adenosine derivative, the second inhibitor, the isolated beetle luciferase, the isolated non-beetle luciferase ATP consuming enzyme, the beetle luciferase substrate, or the substrate for the non-beetle luciferase ATP consuming enzyme, is lyophilized.

51. The kit of claim 44 wherein the second inhibitor is PPi.

52. The kit of claim 44 which further comprises an inhibitor of inorganic pyrophosphatase.

53. A method to identify a concentration of a divalent cation that alters the Kₘ of a luciferase for ATP, comprising:
a) providing the Kₘ of one or more luciferases for ATP in a luminogenic reaction at one or more concentrations of a plurality divalent metals, wherein the Kₘ is optionally in the absence of an inhibitor of ATP; and
b) identifying a concentration of at least one divalent metal and at least one luciferase having a selected Kₘ for ATP in the luminogenic reaction in a linear range for the reaction.
